(19) United States
(12) Patent Application Publication
(10) Pub. No.: US 2010/0068735 A1
(43) Pub. Date: Mar. 18, 2010

(54) MICROFLUIDIC DEVICE INCLUDING UNIT FOR EVALUATING CAPTURE MATERIAL AND METHOD OF EVALUATING CAPTURE MATERIAL

(75) Inventors: Kui-hyun KIM, Suwon-si (KR); Beom-seok LEE, Hwaseong-si (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/556,912

(22) Filed: Sep. 10, 2009

(30) Foreign Application Priority Data
Sep. 12, 2008 (KR) 10-2008-0090499
Aug. 17, 2009 (KR) 10-2009-0075812

(51) Int. Cl. G01N 33/566; C12M 1/34

(52) U.S. Cl. 435/7.92; 435/287.2

(57) ABSTRACT

Provided are a microfluidic device including a unit for evaluating a capture material and a method of evaluating a capture material.

### CROSS-REFERENCE TO RELATED APPLICATIONS

This application claims the benefit of Korean Patent Application Nos. 10-2008-0090499, filed on Sep. 12, 2008 and 10-2009-0075812, filed Aug. 17, 2009, in the Korean Intellectual Property Office, the disclosures of which are incorporated herein in their entirety by reference.

### BACKGROUND

1. Field

One or more embodiments relate to a microfluidic device for evaluating a capture material which is used in biochemical analysis of a sample and a method of evaluating a capture material, allowing assessing functional state of the capture material that is used in biochemical analysis.

2. Description of the Related Art

In general, sample analysis require many steps. For example, to detect a target material in blood, the steps include: collecting blood; storing the collected blood; disrupting a cell in the blood; amplifying a nucleic acid; isolating the target material; and analyzing the target material. Thus, to efficiently perform a sample analysis with many steps, a microfluidic device has been recently used. The microfluidic device has many advantages. For example, a small amount of a reagent is mixed and reacted, and thus costs of the reagent can be minimized, and transfer of a reagent and a sample can easily be controlled using an automated control device, and thus it is more convenient than manual jobs. In addition, the size of microfluidic device is relatively small, and thus an experiment space can be saved.

In general, a sample analysis using the microfluidic device may be performed as follows. First, a liquid sample including a target material is introduced into a sample inlet chamber by fluid driving force, for example, capillary action, air pressure, or centrifugal force. Subsequently, the liquid sample flows into a reaction chamber wherein the liquid sample is mixed with a reaction reagent, such as a labeled antibody. The labels may be fluorescence, electrochemical agent, or other various labels well-known in the art. Then, the liquid sample is subject to a detection of a target material-reagent complex. The detection may be carried out in the reaction chamber or in a separate chamber (“detection chamber”). When a labeled antibody is used as a reaction reagent, an immune complex is formed between the target material and the labeled antibody, and the target material-antibody complex is captured and immobilized in the detection chamber. The captured complex may be measured using a detection device, for example, a fluorescence detector, an optical detector, or an electrical detector.

Reagents used in a sample analysis can be assessed for the functional state to ensure the reliability of the results of the sample analysis. That is, a reagent is reacted in conditions that are the same as or similar to those of the sample analysis in which the reagent is to be used to detect a target material in a sample, and determine whether the activity of the reagent is constant. Also, in a sample analysis using a microfluidic device, reagents to be used in the sample analysis are tested for their activity to determine if the activity thereof is constant. A series of reactions are performed in the microfluidic device, and thus it is difficult to perform a reaction of a control group with respect to a specific reaction. In the microfluidic device, the control group reaction may be carried out for ideally all steps of the sample analysis. However, such is not practical due to its costs, time, and operational difficulties.

### SUMMARY

One or more embodiments include a microfluidic device for efficiently evaluating a capture material or a tracer to assess the functional state of the capture material or the tracer.

One or more embodiments include a method of efficiently evaluating a capture material or a tracer.

Additional aspects will be set forth in part in the description which follows and, in part, will be apparent from the description, or may be learned by practice of the invention.

To achieve the above and/or other aspects, one or more embodiments may include a microfluidic device comprising a chamber comprising a capture material that is bound to a target material, wherein a first chamber of the chamber is in a fluid communication with a chamber comprising a tracer that is bound to the capture material.

One or more embodiments include a microfluidic device for analyzing a target material in a sample, including a chamber (“capture material chamber”) accommodating a capture material that binds the target material, and a chamber (“tracer chamber”) accommodating a tracer that binds the capture material, wherein the capture material chamber is in fluid communication with the trace chamber; and the tracer chamber does not contain the biological sample.

According to another exemplary embodiment, there is provided a method of assessing a functional state of a reagent in a microfluidic device for a biochemical assay of a target material in a sample, the reagent being capable of binding to the target material, the method including: bringing the reagent to be in contact with a tracer that binds the reagent, wherein the reagent is contained in the microfluidic device; detecting interaction between the tracer and the reagent to obtain test detection results; and comparing the test detection results with a control detection results obtained from a control group of a known amount of the tracer to assess the functional state of the reagent, wherein the method of assessing the functional state is performed on the same microfluidic device where the biochemical assay of the target material is performed

### BRIEF DESCRIPTION OF THE DRAWINGS

These and/or other aspects will become apparent and more readily appreciated from the following description of the embodiments, taken in conjunction with the accompanying drawings of which:

FIG. 1 is a diagram illustrating a unit 10 for evaluating a capture material in a microfluidic device according to an embodiment;

FIGS. 2A, 2B, and 2C are diagrams illustrating a tracer 410, a signal generating material 420, and a capture material 400, according to embodiments;

FIG. 3A is a diagram of a disc-shaped sample analyzer in which microfluidic device according to an embodiment is formed, and FIG. 3B is an enlarged diagram of a unit 10 of the microfluidic device of FIG. 3A; and

FIGS. 4A and 4B are graphs showing absorbance results obtained using a signal generating material bound to a tracer in a microfluidic device according to an embodiment.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

A microfluidic device according to an embodiment includes a unit of which at least one of the dimensions of a cross-sectional area, for example, depth, width, length, and diameter is about 0.1 μm to about 20 mm. For example, the unit may be a chamber, a channel, or a reservoir. The microfluidic device may include: a chamber which receives and/or stores a small amount of fluid; a channel through which the fluid flows; a valve and pump which control fluid flow; and a plurality of functional units which carry out a certain operation by receiving the fluid. The fluid is introduced into the microfluidic device, and then may be transferred through the chamber and/or channel by a pump or hydraulic press. The transfer of the fluid may be controlled by a valve. The valve is included in the microfluidic device to open or close the chamber or channel in order to allow a fluid stored in the chamber to be transferred. The valve may be a well-known valve used in a general microfluidic device. For example, the valve may comprise a material that is opened by electromagnetic energy. The valve material may be a phase transition material of which phase is transformed by energy or a thermoplastic resin. The phase transition material may be, for example, a wax or a gel. The valve material may comprise micro heating particles that are dispersed in the phase transition material and generate heat upon absorbing electromagnetic energy. The micro heating particles may be a metal oxide comprising Al₂O₃, TiO₂, Ta₂O₃, Fe₂O₃, Fe₃O₄, or HfO₂, polymer particles, quantum dots, or magnetic beads. The size (e.g., an average or mean diameter) of micro heating particles may vary according to the size of the channel, ranging from nano-meters to micro-meters. The pump may be a unit which can apply a driving force for fluid flow. For example, the pump may be a positive-pressure pump or negative-pressure pump, which applies an air pressure. The valve and/or pump may be installed in the chamber or in a channel connected between the chambers, thereby controlling fluid transfer between the chambers. Thus, the microfluidic device may further include a pump and/or valve that is operably connected to the chamber. The functional units may vary according to a use of the microfluidic device. For example, a unit which can detect the binding between a target material in a liquid sample and a receptor may be formed in a chamber of the chamber.

In addition, the microfluidic device may include a liquid flow control device comprised of a program with consecutive instructions that direct the pump and/or valve to be operated and stopped. For example, the pump and/or valve may be connected to a fluidic control unit or a fluidic control system. The introduction of the fluid, the transfer of the fluid, the storage of the fluid, and the control of the fluid in the microfluidic device are well-known in the art.

The microfluidic device may be formed on a substrate. For example, the substrate may be a centrifugal force-driven substrate. For example, the microfluidic device may be a rotatable disc-shaped device in which an inlet and outlet of a liquid sample, a channel, a chamber, and a valve are formed. Thus, the microfluidic device is formed on a disc-shaped substrate that can rotate around a rotation axis, and at least two chambers, a channel connected between the chambers, and a control unit that can control fluid transfer between the channels, for example, a valve, may be formed on the disc-shaped substrate. The microfluidic device based on centrifugal force rotates around the rotation axis, thereby transferring a fluid from a chamber to another chamber according to the centrifugal force applied to the fluid. In the centrifugal force-based microfluidic device, a plurality of chambers may be radially formed around the center of rotation, and the chambers may be connected by, for example, a channel to be in fluid communication therebetween. In addition, the substrate may be connected to a means for providing a rotation force, for example, a motor or servo motor. The substrate may rotate clockwise or counter clockwise. The substrate may have a variety of shapes, and is not limited to, for example, circular shape or tetragonal shape.

The target material comprises a material to be detected. The target material may comprise a biological material. Examples of the biological material include a nucleic acid, protein or polypeptide, sugar, virus, cell, and cellorganelle. The nucleic acid may be DNA, RNA, or PNA. The cell may be a eukaryotic cell such as plant or animal cell and a prokaryote cell such as bacteria. The biological material may be derived from living organisms, or synthesized or semi-synthesized.

The capture material comprises a material that binds to the target material, for example, a material that can specifically or non-specifically bind the target material. The capture material may be an antibody or antigen, a nucleic acid, an enzyme or substrate, a receptor or ligand, or the like, which binds the target material. The capture material may have a site (a first site) that can bind the target material, and/or a site (a second site) that can bind the tracer, formed at a position that is the same as or different from the binding site described above. The capture material may be immobilized or not immobilized in the chamber. In addition, the capture material may be immobilized on a material with a surface, the material formed in the chamber, for example, a substrate or electrode. Thus, the capture material may bind the target material in a state where the capture material is immobilized in the chamber or not immobilized therein. A method of immobilizing the capture material is well-known in the art. For example, a method of immobilizing a protein on a surface of a substrate by using carboxymethyl-dextran or an avidin-biotin bond is well-known. Also, a method of immobilizing a protein on a surface of a substrate by previously treating the surface of the substrate with a chemical material or a method of using polylysine or calixcrown in order to bind a plurality of unspecific proteins to a surface of a substrate is well-known. A complementary linker used to immobilize an antibody, virus or cell on a surface of a substrate is also well-known.

The tracer binds the target material, and comprises a material allowing detection of the presence or amount of the target material. The detection may be facilitated by the tracer itself or a signal generating material that is lined to the tracer. The tracer may have a site that can bind the target material, and/or a site that can bind the signal generating material, wherein this site is formed at a position that is the same as or different from the binding site described above. The tracer may be the same as or different from the target material. The tracer may be used in a free form (i.e., not immobilized) contained in a fluid. The tracer may be introduced into the microfluidic device, for example, in the chamber when the microfluidic device is in use or ready to use; or may be preloaded and stored the chamber until the use of the microfluidic device. The tracer may be selected from the group consisting of an antibody, a ligand, an enzyme, an enzyme substrate, an enzyme inhibitor, and an antigen; however, the kind of the tracer is not limited thereto.

The tracer may be bound to the signal generating material. The signal generating material is a material that binds the tracer and can generate a signal that can be detected. For example, the signal generating material may be selected from the group consisting of peroxidase, alkaline phosphatase, fluorophore, chemiluminescence probe, gold particles, a radioactive material, latex, and horseradish peroxidase; however, the signal generating material is not limited thereto. The signal generating material may be directly bound to the tracer, or bound to the tracer through a linker. Some of the signal generating materials need more space than others, decreasing an inner space of the microfluidic device available for other materials. For example, when horseradish peroxidase is used as the signal generating material, a substrate is required for detection of a signal from the horseradish peroxidase. In this case, a separate space that can accommodate the substrate, for example, a chamber is required. However, when gold particles or latex particles are used as the signal generating material, a separate material is not required for the detection of the signal.

The microfluidic device includes a chamber (“capture material chamber”) including a capture material that binds a target material. The chamber is in fluid communication with a chamber including a tracer (“tracer chamber”) that binds the capture material. For example, the tracer in the tracer chamber may be transferred to the capture material chamber, where the tracer binds the capture material, via a fluid communication path such as chambers and channels in the microfluidic device.

A detector for detecting the binding between the capture material and the tracer may be operably disposed to be directly connected to the first chamber or disposed adjacent to the first chamber. For example, the detector is directly connected to the capture material chamber, thereby detecting a wave signal generated by the binding between the capture material and the tracer in the capture material chamber. Alternatively, the detector is disposed adjacent to the capture material chamber, thereby detecting a signal generated by the binding between the capture material and the tracer in the capture material chamber to detect the presence and/or amount of the capture material.

The capture material chamber may be in fluid communication with the chamber including a tracer and a separation chamber. The tracer that does not bind the capture material in the capture material chamber may be transferred to the separation chamber. For example, the first chamber in the microfluidic device is in fluid communication with the separation chamber, that is, a sub-chamber, and the tracer is transferred to the capture material chamber, where it binds the capture material, and then the tracer that does not bind the capture material may be transferred to the sub-chamber.

A detector for detecting the tracer flows from the capture material chamber, which is the tracer that does not bind the capture material in the first chamber, may be operably disposed to be directly connected to the separation chamber or disposed adjacent to the separation chamber. For example, the detector measures a signal of a signal generating material that is bound to the unbound tracer in the separation chamber, which flows from the capture material chamber, thereby detecting the presence and/or amount of the capture material. In this case, the detection of the presence and/or amount of the capture material may be performed taking into consideration the initial amounts of the capture material, the tracer, and the binding body of the capture material and the tracer introduced into the microfluidic device.

The detector that is operably disposed with respect to the capture material chamber or the separation chamber may selectively detect a material to be analyzed by binding a biological receptor, which can recognize a specific material, with an electrical or optical transducer to convert the biological interaction and recognition to an electrical or optical signal. A unit including the detector may be formed inside or outside the microfluidic device. The detector may be, for example, selected from the group consisting of a photodetector, such as a fluorescence spectrometer, total internal reflection (TIR), or a surface-enhanced Raman spectrometer (SERS); a wave detector, such as quartz crystal microbalance (QCM), an oscillator circuit, or a frequency counting unit; and an electrical detector, such as an enzyme electrode measuring apparatus, field-effect transistor (FET)-based measuring equipment, an electroactive label measuring apparatus, or an electrochemical measuring apparatus, but is not limited thereto.

The capture material chamber may be comprised of a first chamber where a capture material is present and a second chamber. The second chamber may be in fluid communication with at least one unit selected from the group consisting of a sample storage unit for storing a sample, a cell disruption unit for disrupting a cell, a nucleic acid amplification unit for amplifying a nucleic acid, a target material isolation unit, and an analysis unit for analyzing a target material. In addition, the first chamber of the chamber including a capture material that binds a target material in the microfluidic device may not be in fluid communication with at least one unit selected from the group consisting of a sample storage unit for storing a sample, a cell disruption unit for disrupting a cell, a nucleic acid amplification unit for amplifying a nucleic acid, a target material isolation unit, and an analysis unit for analyzing a target material.

The capture material chamber where a biochemical analysis of a biological sample using a capture material is conducted and the capture material chamber where the evaluation of the capture material is carried out may be the same as or different from each other. That is, the evaluation process of the capture material may be performed in the same chamber where the biochemical analysis of a sample using the capture material is performed, or an independent chamber.

An embodiment provides a method of analyzing a capture material or tracer included in a microfluidic device, the method including: contacting a capture material that binds a target material with a tracer that binds the capture material, wherein the capture material is included in a microfluidic device; detecting interaction between the tracer and the capture material; and comparing the detection results with detection results of a control group to evaluate the capture material or tracer.

The detailed descriptions of the target material, the capture material, the tracer, and the microfluidic device are the same as described above.

In particular, the method of evaluating a capture material or tracer included in a microfluidic device may include contacting the capture material that binds the target material with the tracer that binds the capture material, and the capture material may be included in the microfluidic device.

The contacting of the capture material with the tracer is performed by transferring the tracer that is introduced or previously introduced into the microfluidic device, for example, into a chamber, to a chamber including the capture material. In the contacting process, the tracer may be introduced from a chamber that stores the tracer to be contacted with the capture material. The introduction of the tracer may be performed by opening a valve disposed between the chamber including the tracer and the chamber storing the capture material, that is, an inlet valve. The valve may be disposed in a channel connected between the chamber storing the tracer and the chamber including the capture material, that is, an inlet channel. The contacting of the capture material with the tracer may be performed in a space that is the same as or different from a space where the target material and the capture material contact with each other. In addition, in the contacting process, the tracer may be introduced via a path different from a path through which the target material is introduced to be contacted with the capture material. In this case, the tracer or the target material may be serially or simultaneously contacted with the capture material in the chamber including the capture material.

In particular, the method of the assessing the functional state of a capture material or tracer included in a microfluidic device may include detecting the interaction between the tracer and the capture material. The term “functional state” of the capture material or tracer is used to indicate if the capture material or tracer maintains their activity or potency in a level within a allowable range suitable for a desired biochemical assay of the sample.

The interaction includes a process of detecting the presence or amount of the capture material by the contacting of the capture material with the tracer. For example, the interaction may be, but is not limited to, an immune reaction between an antigen and an antibody, complementary binding between nucleic acids, or binding between a cell and a cell receptor.

The detecting process includes detecting a signal generated in a state where the tracer that is bound to a signal generating material is specifically bound to the capture material, or detecting a physical, chemical, and/or electrical signal generated in a state where the tracer is specifically bound to the capture material. Thus, the interaction between the capture material and the tracer may be directly detected. Alternatively, the detecting process may be performed by indirectly detecting the presence of the tracer in a state where the interaction between the capture material and the tracer does not occur. The indirect detection may be performed after the unbound tracer is introduced into a sub-chamber (e.g., separation chamber) from the chamber including the capture material. The introduction of the tracer into the sub-chamber may be performed by opening a valve disposed between the chamber including the capture material and the sub-chamber, that is, an outlet valve. The valve may be disposed in a channel connected between the chamber including the capture material and the sub-chamber, that is, an outlet channel.

In particular, the method of evaluating a capture material or tracer included in a microfluidic device may include comparing the detection results with detection results of a control group to determine if the capture material and/or tracer maintains their activity at a desired level. The evaluation process may include testing whether a state of the capture material and/or the tracer, for example, binding activity of the capture material and the tracer is maintained constant.

The control group includes a tracer and capture material in a state where they are not transformed or degenerated by temperature, humidity, and/or storage conditions. Thus, the state of the capture material or tracer included in the microfluidic device may be confirmed by comparing detection results of interaction between the capture material and the tracer according to an embodiment with detection results of interaction between the tracer and the capture material of the control group, obtained by contacting the capture material with the tracer. That is, in the detection process, when the interaction between the capture material and the tracer is directly detected, for example, when a measurement value of a signal generated by binding the capture material with the tracer in the microfluidic device is within an allowable range of a signal obtained by biding the capture material with the tracer in the control group, it is concluded that the capture material and/or the tracer in the microfluidic device are not degenerated. Thus, it can be determined that the functional state of the capture material or the tracer is maintained active. Alternatively, in the detection process, when the presence of the tracer is detected in a state where the interaction between the capture material and the tracer does not occur, for example, when a measurement value of a signal generated by the tracer that does not bind the capture material after the binding of the capture material and the tracer in the microfluidic device is within an allowable range of a signal obtained by the tracer that does not bind the capture material after the binding of the capture material and the tracer of the control group, it is concluded that the capture material and/or the tracer in the microfluidic device are not degenerated. Thus, it can be determined that the functional state of the capture material or the tracer is maintained active. The allowable range of the signal refers to a limit in which the functional state of the capture material and/or the tracer is maintained, which can assure reliable analysis results of a sample in the microfluidic device. For example, the allowable limit of the signal may be defined within a certain range having a maximum value and a minimum value of the signal generated by the binding of the capture material and the tracer, or may be defined within an error range of a certain value.

The comparison between the detection results of a test group of capture material or tracer and the detection results of the control group is performed by measuring a relative change in the binding of the capture material and the tracer. For example, when after the capture material immobilized in the chamber including the capture material is bound to the tracer at different ranges of the concentration of tracer and changes in the binding of the capture material and the tracer are measured, a pattern of the change in the concentration of the tracer and a pattern of the change in the binding of the capture material and the tracer are maintained constant, the capture material or the tracer may be determined to be used.

The detection of the control group includes performing the contacting process and the detection process using known amounts of the same materials of known activity, simultaneously or previously, in the same microfluidic device that is used to perform analyzing the test group, or a separate microfluidic device. Alternatively, known data of allowable range of values for the capture material or tracer may be used.

The method of assessing the functional stage of a capture material or tracer included in a microfluidic device may further include detecting interaction between the target material and the capture material in the microfluidic device. Thus, when the functional state of the capture material and/or the tracer is confirmed by comparing with the detection results of the control group, the state of the capture material and/or the tracer used can be determined in the detecting of the interaction between the target material and the capture material. Thus, the detection results of the interaction between the target material and the capture material can be obtained, and moreover, the sample reaction may be analysed.

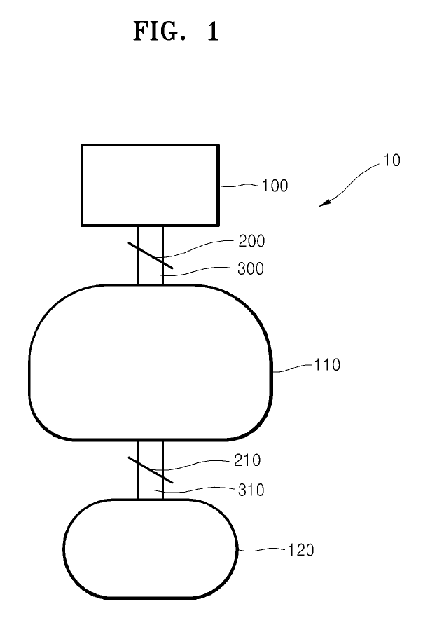

FIG. 1 is a diagram illustrating a unit 10 for evaluating a capture material of a microfluidic device according to an embodiment.

Referring to FIG. 1, the unit 10 includes a tracer chamber 100, a capture material chamber 110, and a sub-chamber 120, which are in fluid communication with one another via channels 300 and 310. The channels 300 and 310 respectively include valves 200 and 210 for controlling fluid flow.

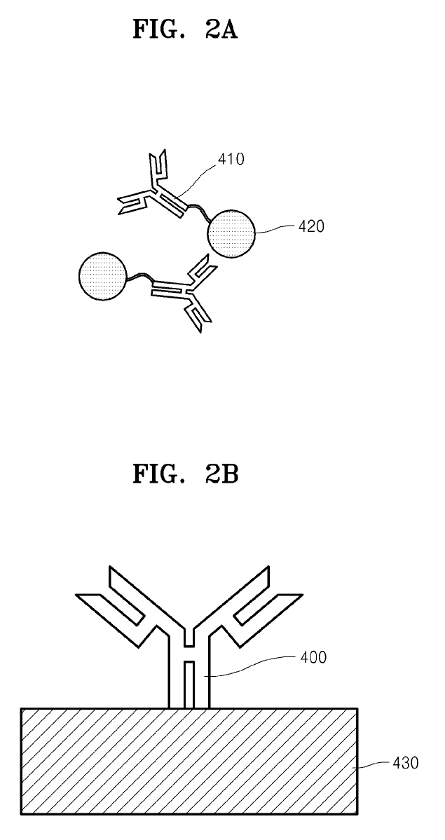

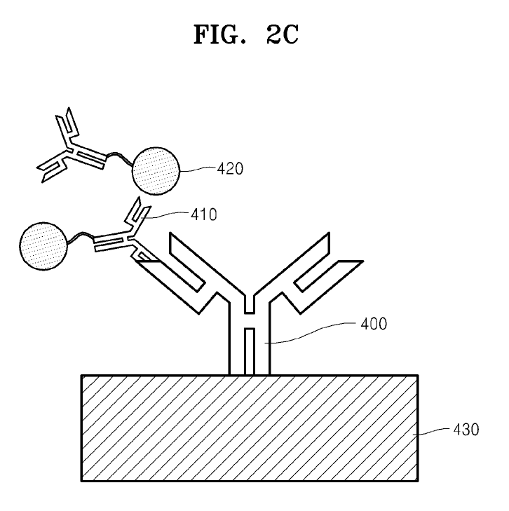

The tracer chamber 100 may include a tracer 410 (refer to FIGS. 2A and 2C) that is bound to a signal generating material 420 (refer to FIGS. 2A and 2C). The signal generating material 420 binds the tracer 410, thereby generating a signal that can be detected. For example, the signal generating material 420 may be gold particles or latex particles. The signal generating material 420 may be directly coupled to the tracer 410, or may be coupled to the tracer 410 through a linker, which is an agent that specifically binds the tracer 410 and the signal generating material 420. The tracer 410 binds the target material, and is a material that can detect the presence or amount of the target material alone or together with the signal generating material 420. For example, the tracer 410 may be an antibody which can specifically bind an antigen. The tracer 410 may be previously loaded into and stored in the tracer chamber 100 until its use, or may be introduced into the tracer chamber 100 when used. The tracer 410, in its free from (i.e., un-immobilized form) may exist in a liquid. The capture material chamber 110 includes a capture material 400 (refer to FIGS. 2B and 2C). The capture material may be a material that can specifically or non-specifically binds the target material, for example, an antibody that can specifically bind an antibody. The capture material 400 may be immobilized. For example, the capture material 400 is immobilized on a surface of inner walls of the capture material chamber 110. After the contact between the tracer 410 and the capture material 400, the tracer 410 that does not bind the capture material 400 in the capture material chamber 110 (“unbound tracer 410”) is introduced into the sub-chamber 120. In FIG. 1, the unit may not include the sub-chamber 120.

The unit 10 includes an inlet channel 300 connected between the tracer chamber 100 and the capture material chamber 110 and an outlet channel 310 connected between the capture material chamber 110 and the sub-chamber 120. The tracer 410 that is linked to the signal generating material 410 is introduced into the capture material chamber 110 via the inlet channel 300, and unbound tracer 410, after the contact with the capture material, is introduced into the sub-chamber 120 via the outlet channel 310.

The unit 10 includes an inlet valve 200 that controls the flow of fluid introduced into the capture material chamber 110 and is disposed at the inlet channel 300 and an outlet valve 210 that controls the flow of fluid introduced into the sub-chamber 120 and is disposed at the outlet channel 310. When the inlet valve 200 is closed, the tracer chamber 100 is sealed, and when the inlet valve 200 is opened, the tracer 410 linked to the signal generating material 420 in the tracer chamber 100 can be transferred to the capture material chamber 110. Interaction between the capture material 400 and the tracer 410 occurs in the capture material chamber 110. In addition, when the inlet valve 200 and the outlet valve 210 are closed, the capture material chamber 110 is closed, on the other hand, when the outlet valve 210 is opened, the tracer 410 linked to the signal generating material 420, which does not bind the capture material 400 in the capture material chamber 110 may be transferred to the sub-chamber 120.

In the unit 10, the valves 200 and 210 may be connected to a liquid flow control device storing a program including instructions to operate the valves 200 and 210. For example, the valves 200 and 210 may be connected to a fluidic control system, thereby controlling the flow of liquid.

In unit 10, a detector (not shown) for detecting a signal generated by the signal generating material 420 may be operably disposed with respect to the capture material chamber 110 and/or the sub-chamber 120. The detector may be disposed inside or outside the unit 10. The detector may be a photodetector, such as a fluorescence spectrometer, or a wave detector, such as an oscillator circuit. Thus, the detector may detect the interaction between the tracer 410 and the capture material 400 in the capture material chamber 110 or detect the tracer 410 in the sub-chamber 120.

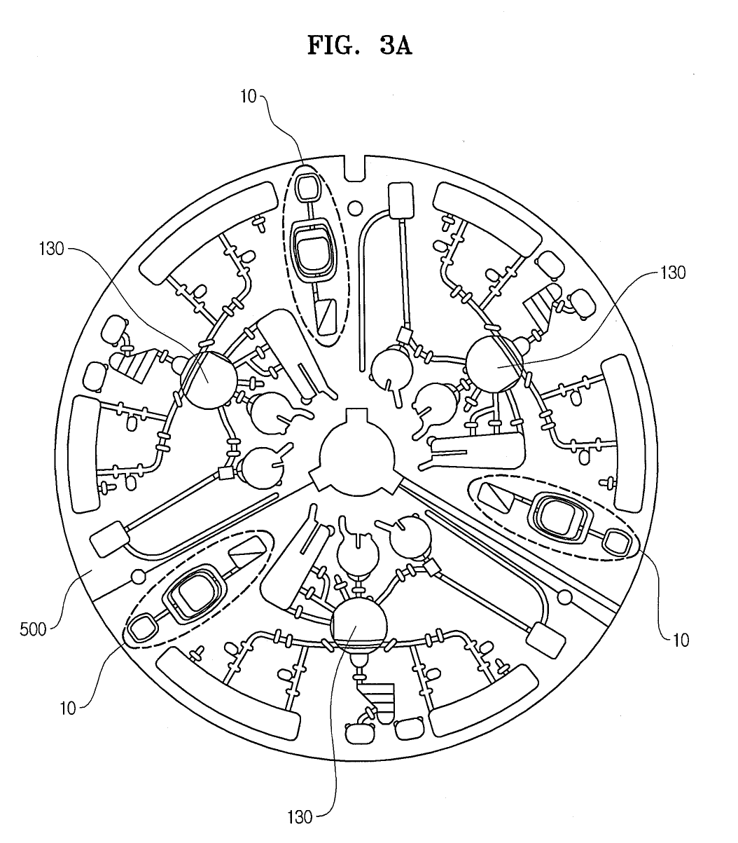

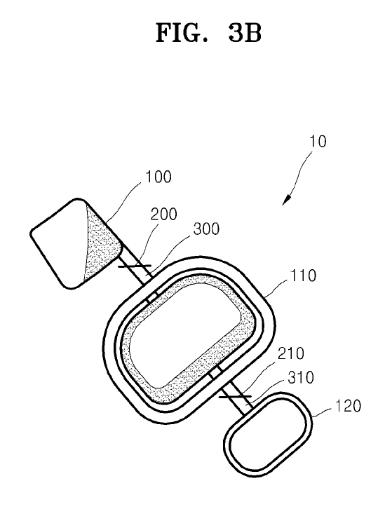

The unit 10 may be disposed on a rotatable disc-shaped substrate 500 (refer to FIG. 3A) that is driven by centrifugal force. Thus, the unit 10 rotates around a rotation axis connected to a motor, thereby transferring a fluid from a chamber to another chamber according to the centrifugal force applied to the fluid.

FIGS. 2A, 2B, and 2C are diagrams illustrating a tracer 410, a signal generating material 420, and a capture material 400, according to embodiments.

FIG. 2A is a diagram illustrating a tracer 410 and a signal generating material 420 bound to the tracer 410, according to an embodiment. The tracer 410 may be goat anti-mouse IgG, and the signal generating material 420 may be gold particles or latex particles. The tracer 410 coupled to the signal generating material 420 may be previously loaded in the tracer chamber 100 (refer to FIG. 1) when a microfluidic device is prepared, or may be introduced into the tracer chamber 100 when used. In FIG. 2A, the tracer 410 is coupled to the signal generating material 420, but the tracer 410 may not be coupled to the signal generating material 420 depending on the detection method (for example, when a surface acoustic wave sensor is used to detect the interaction between the tracer 410 and the target material or capture material).

FIG. 2B is a diagram illustrating a state where the capture material 400 is immobilized on a substrate 430. The substrate 430 may be a surface of inner walls of the capture material chamber 110.

FIG. 2C is a diagram illustrating a state where the tracer 410 coupled to the signal generating material 420 is bound to the capture material 400 immobilized on the surface of the inner wall of the capture material chamber 110. The capture material 400 has a site that can bind the target material, and/or a site that can bind the tracer 410, which is formed in the same position or different from the binding site described above.

FIG. 3A is a diagram of a microfluidic device 500 including a unit 10 for evaluating a capture material, according to an embodiment, and FIG. 3B is an enlarged diagram of the unit 10 of the microfluidic device of FIG. 3A.

Referring to FIG. 3A, the microfluidic device 500 includes a capture material chamber 130 (second chamber) that is separated from a capture material chamber 110 (first chamber) (110 of the unit 10 in FIGS. 3A and 3B). Thus, the second chamber 130 may be used for analyzing a target material in a sample, and, the first chamber 110 may be used for evaluating a capture material. The capture material in the first chamber 110 may be immobilized and sealed therein in the same manner as in the process of immobilizing and sealing the capture material in the second chamber 130 in the preparation of the microfluidic device.

The microfluidic device is formed on a circular disc-shaped substrate, and may be driven by centrifugal force. The microfluidic device may include an inlet for a liquid sample, a channel for transferring the liquid sample and a reagent for analyzing the liquid sample, a chamber for storing the sample and the reagent, a chamber including an analysis unit for detecting the target material, and a valve for controlling flow of the sample and the reagent. Thus, the microfluidic device rotates around a rotation axis, thereby transferring the liquid sample from a chamber to another chamber according to the centrifugal force applied to the liquid sample, which can be used in detecting the target material in the liquid sample.

Referring to FIG. 3B, the unit 10 includes a tracer chamber 100, a capture material chamber 110, and a sub-chamber 120, which are in fluid communication with one another via channels 300 and 310. The channels 300 and 310 include valves 200 and 210 that can control the fluid flow. The tracer chamber 100 includes a tracer, and the capture material chamber 110 includes a capture material immobilized therein.

### EXAMPLE 1

### Confirmation of Functional States of Capture Material and Tracer by Using Tracer to which Gold Particles are Coupled

The circular disc-shaped microfluidic device for analyzing a sample of FIG. 3A was used.

An inner wall of a capture material chamber in the microfluidic device was coated with goat anti-mouse IgG, and 100 μl (½×) (Experiment 1) of a solution including mouse IgG to which gold particles were coupled was introduced into the tracer chamber and then the tracer chamber was sealed. The size of gold particles was limited to about 5 nm to about 1,000 nm.

Blood was injected into a liquid sample inlet in the microfluidic device, and a target material in the blood was detected and/or analyzed by controlling the rotation of the microfluidic device by using a fluidic control system and a valve. After the above detection and/or analysis processes were performed, the functional state of the goat anti-mouse IgG or the mouse IgG was confirmed as follows.

In the confirmation process, first, by opening an inlet valve and rotating the microfluidic device, 100 μl of the solution including the mouse IgG to which gold particles were coupled, which was sealed in the tracer chamber, was transferred to the capture material chamber.

Then, the microfluidic device was stopped from rotating, and incubated for 20 minutes so that the mouse IgG transferred to the capture material chamber was contacted with the goat anti-mouse IgG to be bound to each other.

Then, in a state where the inlet valve was closed, an outlet valve was opened, and mouse IgG that did not bind the goat anti-mouse IgG was transferred to a sub-chamber by fully rotating the microfluidic device until unbound mouse IgG coupled to gold particles was removed from the capture material chamber. This separation may be performed using known methods.

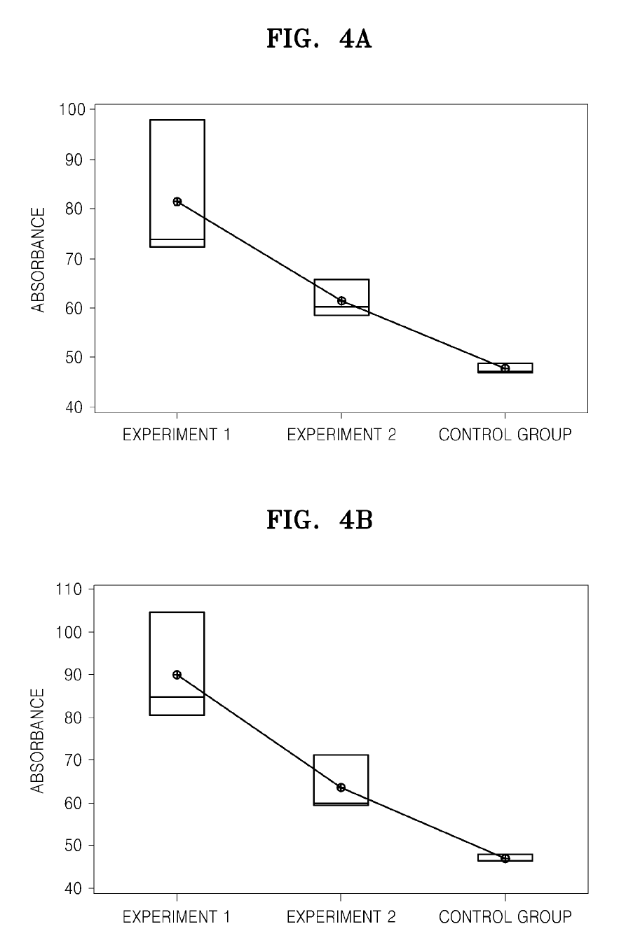

Then, absorbance of a signal generated by the unbound mouse IgG transferred to the sub-chamber was measured at a wavelength of 550 nm by using an ELISA reader. The processes described above were consecutively performed on mouse IgG coupled to gold particles at a concentration of ⅛× (Experiment 2) and 0× (control group) in different chambers, and absorbance of each group was measured. A change in the absorbances is illustrated in FIG. 4A (P value=0.009).

As can be seen in FIG. 4A, when the concentration of the tracer was smaller, that is, when gold particles were bound to mouse IgG, the absorbance decreased. Thus, states of the capture material, that is, goat anti-mouse IgG and the tracer, that is, mouse IgG, can be determined to be used. That is, when the concentration of the capture material and the tracer was smaller, there was little binding activity between the capture material and the tracer, and thus it can be determined that the activity was maintained constant. FIG. 4A is a graph showing measurement results of absorbances of Experimental Group 1, Experimental Group 2, and Control Group.

### EXAMPLE 2

### Confirmation of Functional States of Capture Material and Tracer by Using Tracer to which Latex Particles are Bound

The circular disc-shaped microfluidic device for analyzing a sample of FIG. 3A was used.

An inner wall of a capture material chamber in the microfluidic device was coated with goat anti-mouse IgG, and 100 μl (¼×) (Experiment 1) of a solution including mouse IgG to which latex particles were coupled was introduced into a tracer chamber and then the tracer chamber was sealed. The size of latex particles was limited to about 5 nm to about 1,000 nm.

Blood was injected into a liquid sample inlet in the microfluidic device, and a target material in the blood was detected and/or analyzed by controlling the rotation of the microfluidic device by using a fluidic control system and a valve. After the above detection and/or analysis processes were performed, a state of the goat anti-mouse IgG or the mouse IgG was confirmed.

In the confirmation process, first, by opening an inlet valve and rotating the microfluidic device, 100 μl of the solution including the mouse IgG to which latex particles were coupled, which was stored in the tracer chamber with being sealed, was transferred to the capture material chamber.

Then, the microfluidic device was stopped from rotating, and incubated for 20 minutes so that the mouse IgG transferred to the capture material chamber was contacted with the goat anti-mouse IgG to be bound to each other.

Then, in a state where the inlet valve was closed, an outlet valve was opened, and unbound mouse IgG was transferred to a sub-chamber by fully rotating the microfluidic device until it was removed from the capture material chamber.

Then, absorbance of a signal generated by the unbound mouse IgG transferred to the sub-chamber was measured at a wavelength of 600 nm by using an ELISA reader. The processes described above were consecutively performed on mouse IgG to which latex particles were coupled having a concentration of ⅛× (Experiment 2) and 0× (control group) in different chambers, and absorbance of each group was measured. A change in the absorbances is illustrated in FIG. 4B (P value=0.002).

As can be seen in FIG. 4B, when the concentration of the tracer (mouse IgG coupled to latex particles) was smaller, the absorbance was small. Thus, the functional states of the capture material, that is, goat anti-mouse IgG and the tracer, that is, mouse IgG, can be confirmed to be suitable for use in the biochemical assay. That is, when the concentration of the capture material and the tracer was small, there was little binding activity between the capture material and the tracer, and thus it can be determined that the activity is maintained constant. FIG. 4B is a graph showing measurement results of absorbances of Experimental Group 1, Experimental Group 2, and Control Group.

As described above, according to the one or more of the above embodiments, a capture material or tracer in a microfluidic device can be efficiently evaluated.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A microfluidic device for analyzing a target material in a sample, comprising
a first chamber (“capture material chamber”) accommodating a capture material that binds the target material, and
a second chamber (“tracer chamber”) accommodating a tracer that binds the capture material,
wherein the capture material chamber is in fluid communication with the tracer chamber so that the tracer flows from the tracer chamber to the capture material chamber where the tracer is brought to be in contact with the capture material; and
the tracer chamber does not contain the biological sample.

2. The microfluidic device of claim 1, wherein the tracer is the same as or different from the target material and provided in the tracer chamber at a known concentration.

3. The microfluidic device of claim 1, wherein the capture material is immobilized.

4. The microfluidic device of claim 1, which further comprise a third chamber (“separation chamber”) receiving a tracer which is discharged from the capture material chamber and is not bound to the capture material,
wherein the capture material chamber is in fluid communication with the separation chamber.

5. The microfluidic device of claim 4, which further comprises a detector for detecting the tracer in the separation chamber, said detector being operably disposed with respect to the separation chamber.

6. The microfluidic device of claim 1, wherein the capture material chamber comprises an auxiliary chamber that is in fluid communication with at least one unit selected from the group consisting of a sample storage unit for storing a sample, a cell disruption unit for disrupting a cell, a nucleic acid amplification unit for amplifying a nucleic acid, a target material isolation unit, and an analysis unit for analyzing a target material, and wherein the units are disposed on the microfluidic device.

7. The microfluidic device of claim 6, wherein the auxiliary chamber is separated from the capture material chamber.

8. The microfluidic device of claim 1, wherein the tracer is coupled to a signal generating material that is gold particles or latex particles.

9. The microfluidic device of claim 1, which further comprise a detector for detecting binding between the capture material and the tracer in the capture material chamber, wherein the detector is operably disposed with respect to the first chamber.

10. The microfluidic device of claim 1, which is formed on a centrifugal force-driven substrate.

11. A method of evaluating a material in a microfluidic device, the method comprising:
contacting a capture material that binds a target material in a sample with a tracer that binds the capture material, wherein the capture material is included in the microfluidic device;
detecting interaction between the tracer and the capture material; and
comparing the detection results with detection results of a control group of a known amount of the tracer to evaluate at least one selected from the group consisting of the capture material and the tracer.

12. The method of claim 11, further comprising contacting the capture material with the target material and detecting interaction between the target material and the capture material in the microfluidic device.

13. The method of claim 12, wherein the contacting of the capture material with the tracer is performed in a space that is the same as or different from a space where the contact of the target material and the capture material occurs.

14. The method of claim 11, wherein, in the contacting of the capture material with the tracer, the tracer is introduced from a chamber that stores the tracer.

15. The method of claim 11, wherein the detecting of the interaction between the tracer and the capture material is performed by assaying the amount of the tracer that is bound to the capture material.

16. The method of claim 11, wherein the detecting of the interaction between the tracer and the capture material is performed by detecting the presence of the tracer that is not bound to the capture material.

17. The method of claim 15, wherein, in the comparing process, when a detection value of the capture material or the tracer is within an allowable range obtained by detection of the control group, the functional state of the capture material or the tracer is determined to be maintained active.

18. The method of claim 11, wherein the detection of the control group is performed such that the contacting and detecting operations are performed in a microfluidic device that is separated from the microfluidic device where the contacting and detection of the capture or tracer material is performed.

19. The method of claim 11, wherein the microfluidic device is formed on a centrifugal force-driven substrate.

20. A microfluidic device for analyzing a target material in a sample, comprising
a unit for performing analysis of the target material by detecting an interaction of the target material and a first capture material that binds the target material,
a first chamber (“capture material chamber”) accommodating a second capture material that binds the target material, and
a second chamber (“tracer chamber”) accommodating a tracer that binds the second capture material,
wherein the capture material chamber is in fluid communication with the tracer chamber so that the tracer flows from the tracer chamber to the capture material chamber where the tracer is brought to be in contact with the capture material;
the tracer chamber does not contain the biological sample; and
the first capture material and the second capture material are the same material.

21. A method of assessing a functional state of a reagent in a microfluidic device for a biochemical assay of a target material in a sample, the reagent being capable of binding to the target material, the method comprising:
bringing the reagent to be in contact with a tracer that binds the reagent, wherein the reagent is contained in the microfluidic device;
detecting interaction between the tracer and the reagent to obtain test detection results; and
comparing the test detection results with a control detection results obtained from a control group of a known amount of the tracer to assess the functional state of the reagent,
wherein the method of assessing the functional state is performed on the same microfluidic device where the biochemical assay of the target material is performed.

22. The method of claim 16, wherein, in the comparing process, when a detection value of the capture material or the tracer is within an allowable range obtained by detection of the control group, the functional state of the capture material or the tracer is determined to be maintained active.
